(19) United States
(12) Patent Application Publication
(10) Pub. No.: US 2023/0228830 A1
(43) Pub. Date: Jul. 20, 2023

(54) APPARATUS AND METHOD FOR OBTAINING IMAGE INFORMATION ON TARGET USING MAGNETIC PARTICLE IMAGING

(71) Applicant: ELECTRONICS AND TELECOMMUNICATIONS RESEARCH INSTITUTE, Daejeon (KR)

(72) Inventor: Tae Yi KIM, Daejeon (KR)

(73) Assignee: ELECTRONICS AND TELECOMMUNICATIONS RESEARCH INSTITUTE, Daejeon (KR)

(21) Appl. No.: 18/156,920

(22) Filed: Jan. 19, 2023

(30) Foreign Application Priority Data
Jan. 20, 2022 (KR) 10-2022-0008751
Jan. 19, 2023 (KR) 10-2023-0007881

(51) Int. Cl. G01R 33/12; A61B 5/0515; A61B 5/00

(52) U.S. Cl. CPC G01R 33/1276; A61B 5/0515; A61B 5/0042

(57) ABSTRACT

The present disclosure provides an apparatus for obtaining image information on a target using magnetic particle imaging (MPI), the apparatus including: a magnetic field generating means including a first magnetic member and a second magnetic member; and at least one processor operably connected to the magnetic field generating means, wherein the at least one processor is configured to cause the magnetic field generating means to form magnetic fields in an ambient space of the target according to a predetermined rule, determine, as a field free line (FFL), a position corresponding to a point, a line, or a plane at which strength of the magnetic fields in the ambient space is less than a threshold value, provide a first control command for the magnetic field generating means such that the field free line moves along a predetermined path, identify the field free line changed in response to movement of the magnetic field generating means according to the first control command, and generate the image information on the target on the basis of the field free line changed, and the magnetic fields generated from the first magnetic member and the second magnetic member are asymmetric with respect to the target.

### CROSS REFERENCE TO RELATED APPLICATION

The present application claims priority to Korean Patent Application No. 10-2022-0008751, filed Jan. 20, 2022 and Korean Patent Application No. 10-2023-0007881, filed Jan. 19, 2023, the entire contents of which is incorporated herein for all purposes by this reference.

### BACKGROUND OF THE INVENTION

### Field of the Invention

The present disclosure relates to a technology for obtaining image information on a target using magnetic particle imaging (MPI). More particularly, the present disclosure relate to a technology for extending the field of view (FoV) of an image generated from an MPI image.

### Description of the Related Art

Magnetic particle imaging (MPI) using superparamagnetic iron oxide nano particles (SPIONs) is a technique of medical imaging equipment that replaces positron emission tomography (PET), and is a next-generation medical imaging technique that has undergone much research and many developments since the principle was announced in 2005.

In order to make MPI equipment capable of extension into a 3D space, it is necessary to make a field free line (FFL) in which the strength of a magnetic field at a point, line, and plane in space is close to 0 (zero).

The steeper the slope of the magnetic field in the FFL, the greater the resolution. Therefore, making this region in space is the most important part of MPI.

Methods proposed so far can be broadly divided into two types: a method using an electromagnet, and a method using a permanent magnet.

The FFL is moved to the left and right of a sample to be photographed and is rotated a 360 degree angle to obtain a sinogram. Inverse transformation is performed on the obtained sinogram to obtain an MPI image, and for this reason, the region across which the FFL passes is the FoV in MPI.

Making an FFL with a wide region and a high magnetic field gradient (equal to or greater than 2 T/m) is a very important goal in the course of the current global MPI development. In research on the development of an MPI system based on a mechanically moving FFL generator, the overall size of an FFL generator including a permanent magnet needs to be large in order to widen the FoV with the FFL generator. As the overall size of the FFL generator is large, it is difficult to increase the magnetic field gradient of the FFL generator. In other words, in the development of an FFL generator, it is difficult to widen the FoV and increase a magnetic field gradient (improvement in MPI resolution) simultaneously.

The foregoing is intended merely to aid in the understanding of the background of the present disclosure, and is not intended to mean that the present disclosure falls within the purview of the related art that is already known to those skilled in the art.

### SUMMARY OF THE INVENTION

The present disclosure is directed to providing a method and an apparatus for extending the FoV of image information obtained through MPI.

According to an embodiment of the present disclosure, there is provided an apparatus for obtaining image information on a target using magnetic particle imaging (MPI), the apparatus including: a magnetic field generating means including a first magnetic member and a second magnetic member; and at least one processor operably connected to the magnetic field generating means, wherein the at least one processor is configured to cause the magnetic field generating means to form magnetic fields in an ambient space of the target according to a predetermined rule, determine, as a field free line (FFL), a position corresponding to a point, a line, or a plane at which strength of the magnetic fields in the ambient space is less than a threshold value, provide a first control command for the magnetic field generating means such that the field free line moves along a predetermined path, identify the field free line changed in response to movement of the magnetic field generating means according to the first control command, and generate the image information on the target on the basis of the field free line changed, and the magnetic fields generated from the first magnetic member and the second magnetic member are asymmetric with respect to the target.

In addition, the predetermined path may be a first path formed asymmetrically with respect to the target.

In addition, the at least one processor may be further configured to provide a second control command for control such that positions of the first magnetic member and the second magnetic member are exchanged or strengths of the magnetic fields generated by the first magnetic member and the second magnetic member are exchanged, and the field free line changed may be identified in response to movement of the magnetic field generating means according to the second control command.

In addition, the magnetic fields generated from the first magnetic member and the second magnetic member may be generated symmetrically with respect to the target, and the predetermined path may be a second path formed symmetrically with respect to the target.

In addition, the at least one processor may be configured to identify a final field free line on the basis of the field free line moving along the first path or the field free line moving along the second path, and generate the image information on the target on the basis of the final field free line.

According to an embodiment of the present disclosure, there is provided an operation method of an apparatus for obtaining image information on a target using magnetic particle imaging (MPI), the operation method including causing a magnetic field generating means to form magnetic fields in an ambient space of the target according to a predetermined rule; determining, as a field free line (FFL, a position corresponding to a point, a line, or a plane at which strength of the magnetic fields in the ambient space is less than a threshold value; providing a first control command for the magnetic field generating means such that the field free line moves along a predetermined path; identifying the field free line changed in response to movement of the magnetic field generating means according to the first control command; and generating the image information on the target on the basis of the field free line changed, wherein the magnetic fields respectively generated from a first magnetic member and a second magnetic member that the magnetic field generating means includes are asymmetric with respect to the target.

In addition, the predetermined path may be a first path formed asymmetrically with respect to the target.

In addition, the operation method may further include providing a second control command for control such that positions of the first magnetic member and the second magnetic member are exchanged or strengths of the magnetic fields generated by the first magnetic member and the second magnetic member are exchanged, wherein the field free line changed may be identified in response to movement of the magnetic field generating means according to the second control command.

In addition, the magnetic fields generated from the first magnetic member and the second magnetic member may be generated symmetrically with respect to the target, and the predetermined path may be a second path formed symmetrically with respect to the target.

In addition, the operation method may further include: identifying a final field free line on the basis of the field free line moving along the first path or the field free line moving along the second path; and generating the image information on the target on the basis of the final field free line.

According to an embodiment of the present disclosure, there is provided an apparatus for obtaining image information on a target using magnetic particle imaging (MPI), the apparatus including: a first magnetic field generating means provided symmetrically with respect to the target; a second magnetic field generating means provided asymmetrically with respect to the target; and at least one processor operably connected to the first magnetic field generating means and the second magnetic field generating means, wherein the at least one processor is configured to cause the first magnetic field generating means and the second magnetic field generating means to form magnetic fields in an ambient space of the target according to a predetermined rule, determine, as field free lines (FFLs), positions corresponding to points, lines, or planes at which strengths of the magnetic fields in the ambient space are less than a threshold value, the field free lines including a first field free line and a second field free line respectively corresponding to the first magnetic field generating means and the second magnetic field generating means, identify, as a first region and a second region, respective regions that the first field free line and the second field free line occupy as moving along respective predetermined paths, and generate the image information on the target on the basis of the identified first region and the identified second region.

In addition, the predetermined paths may include: a first path formed symmetrically with respect to the target; and a second path formed asymmetrically with respect to the target, wherein the first path may correspond to the first region, and the second path may correspond to the second region.

In addition, the at least one processor may be configured to identify an overlapping region between the first region and the second region, and exclude the overlapping region in order to generate the image information on the target on the basis of the identified first region and the identified second region.

According to an embodiment of the present disclosure, there is provided an operation method of an apparatus for obtaining image information on a target using magnetic particle imaging (MPI), the operation method including causing a first magnetic field generating means and a second magnetic field generating means to form magnetic fields in an ambient space of the target according to a predetermined rule, the first magnetic field generating means being provided symmetrically with respect to the target and the second magnetic field generating means being provided asymmetrically with respect to the target; determining, as field free lines (FFLs), positions corresponding to points, lines, or planes at which strengths of the magnetic fields in the ambient space are less than a threshold value, the field free lines including a first field free line and a second field free line respectively corresponding to the first magnetic field generating means and the second magnetic field generating means; identifying, as a first region and a second region, respective regions that the first field free line and the second field free line occupy as moving along respective predetermined paths; and generating the image information on the target on the basis of the identified first region and the identified second region.

In addition, the predetermined paths may include: a first path formed symmetrically with respect to the target; and a second path formed asymmetrically with respect to the target, wherein the first path may correspond to the first region, and the second path may correspond to the second region.

In addition, the operation method may further include identifying an overlapping region between the first region and the second region, and the generating of the image information on the target on the basis of the identified first region and the identified second region further includes excluding the overlapping region.

According to the present disclosure, the FoV of image information obtained through MPI can be extended.

### BRIEF DESCRIPTION OF THE DRAWINGS

The above and other objectives, features, and other advantages of the present disclosure will be more dearly understood from the following detailed description when taken in conjunction with the accompanying drawings, in which:

FIG. 1 is a flowchart illustrating an operation of an apparatus according to an embodiment of the present disclosure;

FIG. 2 is a flowchart illustrating another operation of an apparatus according to an embodiment of the present disclosure;

FIG. 3 is a configuration diagram illustrating a magnetic field generating means included in an apparatus according to an embodiment of the present disclosure;

FIG. 4 is a configuration diagram illustrating a magnetic field generating means included in an apparatus according to an embodiment of the present disclosure;

FIG. 5 is a configuration diagram illustrating an operation of generating image information with an extended FoV by an apparatus according to an embodiment of the present disclosure; and

FIGS. 6A to 6D are diagrams illustrating image information generated by an apparatus according to an embodiment of the present disclosure.

### DETAILED DESCRIPTION OF THE INVENTION

The expression “according to some embodiments” or “according to an embodiment” used throughout the specification does not necessarily indicate the same embodiment.

Some embodiments of the present disclosure may be described into functional block components and various processing steps. Some or all of the functional blocks may be realized as any number of hardware and/or software components performing specific functions. For example, functional blocks of the present disclosure may be realized by one or more microprocessors or by circuit components for a predetermined function. In addition, for example, the functional blocks of the present disclosure may be realized in various programing or scripting languages. The functional blocks may be realized as an algorithm running on one or more processors. In addition, the present disclosure may employ conventional techniques for electronic environment setting signal processing and/or data processing. The terms “mechanism”, “element”, “means”, and “component” may be widely used, and are not limited to mechanical and physical components.

Furthermore, connecting lines or connecting members between constituent elements shown in the drawings are merely illustrative of functional connections and/or physical or circuit connections. Connections between constituent elements may be represented by various alternative or additional functional connections, physical connections, or circuit connections in a practical device.

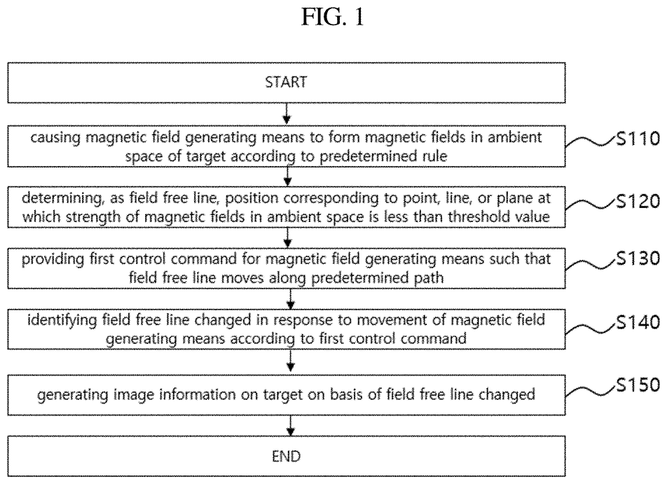

FIG. 1 is a flowchart illustrating an operation of an apparatus according to an embodiment of the present disclosure.

The steps to be described below are not limited to being performed in the described order unless there is a special circumstance in which the steps must be performed in that order bemuse of a causal relationship between the listed steps.

In step S110, the apparatus causes a magnetic field generating means to form magnetic fields in an ambient space of a target according to a predetermined rule.

The apparatus may include at least one magnetic field generating means. Preferably, a plurality of magnetic field generating means may be included. Specifically, when a plurality of magnetic field generating means are present in the apparatus, the apparatus may form two distinguished regions each involving at least one magnetic field generating means. The two distinguished regions may be referred to as a first region and a second region below.

A magnetic field generating means may be a permanent magnet or an electromagnet. Different magnetic fields may be formed according to predetermined rules based on property, arrangement, or surrounding environment of magnetic field generating means.

For example, the apparatus may control magnetic field generating means such that magnetic fields generated in the first region and the second region are symmetric to each other. More specifically, in the case in which the magnetic field generating means are permanent magnets, the apparatus includes the same magnet provided in the first region and the second region. In the case in which the magnetic field generating means are electromagnets, the currents flowing through the electromagnets or the number of windings are adaptively changed. Accordingly, the magnetic fields having the same strength may be generated. The expression that the magnetic fields are symmetric to each other may mean that magnetic force lines generated from the respective magnetic field generating means are formed symmetrically with respect to any reference point or reference line.

In another example, the apparatus may control magnetic field generating means such that magnetic fields generated in the first region and the second region are not symmetric to each other. More specifically, the apparatus may include permanent magnets of different types or sizes provided in the first region and the second region, respectively, or the strength of the currents flowing through electromagnets or the number of windings may be changed. Accordingly, the magnetic fields generated in the respective regions may be formed asymmetrically to each other.

The apparatus may be realized to obtain a detection signal corresponding to MPI information corresponding to an object. Herein, the object may be referred to as the target. For example, if the apparatus is an MPI measurement apparatus, the apparatus may cause a magnetic field generating means to form symmetric or asymmetric magnetic fields around the target (e.g., a human body).

In step S120, the apparatus determines, as a field free line (FFL, a position corresponding to a point, line, or plane at which the strength of the magnetic fields in the ambient space is less than a threshold value.

A field free line may refer to a line including a point at which the strength of the magnetic fields converges to 0 in a space in which the magnetic fields are formed. In the present disclosure, a field free line may be used as a term including a point or a plane including the point at which the strength of the magnetic fields converges to 0.

The expression that the strength of the magnetic fields converges to 0 may mean that the strength of the magnetic fields measured at a particular point is less than a predetermined reference value (threshold value). For example, when the strength of the magnetic fields measured at a particular point (x, y, z) in the ambient space for the apparatus is 0.5 A/m and a predetermined reference value is 1 A/m, the point (x, y, z) may be any point included in a field free line.

The apparatus may measure the strength of the magnetic fields at any point in the ambient space of the target, or may obtain information on the measured strength of the magnetic fields, thereby determining the position of a field free line in the ambient space.

Herein, the determined position of the field free line may be defined considering a relation with the target. More specifically, the apparatus may identify the position of a field free line relative to the target. For example, the apparatus may identify whether the field free line is positioned at the exact center of the target or may identify the angle or distance at or by which the field free line deviates from the center of the target.

Herein, the position of the field free line relative to the target measured by the apparatus may be referred to as an initial position. According to an embodiment, the apparatus may change the initial position of the field free line by changing the property, arrangement, or surrounding environment of a magnetic field generating means.

In step S130, the apparatus provides a first control command for a magnetic field generating means such that the field free line moves along a predetermined path.

The first control command may be generated through a processor included in the apparatus and may be transmitted to a magnetic field generating means. A magnetic field generating means may generate changed magnetic fields in response to receiving the first control command. The operation of generating the changed magnetic fields by a magnetic field generating means may be realized by changing a physical position of a permanent magnet of the magnetic field generating means or by changing the strength of a current flowing through an electromagnet.

The generating of the changed magnetic fields by a magnetic field generating means may change the position or size of the field free line formed by the apparatus. More specifically, when a magnetic field generating means moves from a first position to a second position according to the first control command, the position of the field free line may be changed from the initial position. Herein, the first position may refer to a physical position of the magnetic field generating means when the field free line is at the initial position. For example, when a magnetic field generating means including a permanent magnet moves from the left to the right, the field free line positioned at the exact center of the target may move to the right.

The predetermined path may be a path along which the field free line moves with respect to the target. The apparatus moves the field free line along the predetermined path to obtain a detection signal for generating MPI information on the target. The predetermined path may be determined as being symmetric or asymmetric with respect to a predetermined reference region. Herein, the reference region may be a region that the target occupies.

Predetermined paths may be divided into a first path asymmetric with respect to the reference region and a second path symmetric with respect to the reference region. When a magnetic field generating means generates asymmetric magnetic fields, the field free line moves along the first path. When a magnetic field generating means generates symmetric magnetic fields, the field free line moves along the second path.

In step S140, the apparatus identifies the changed field free line in response to the movement of a magnetic field generating means according to the first control command.

When a magnetic field generating means moves according to the first control command and the field free line is changed, the apparatus may identify either a final position or a moving path of the changed field free line or both. Herein, the first control command may be a command for a magnetic field generating means to generate changed magnetic fields during a time period in which the field free line and the region that the target occupies overlap.

The operation of identifying the changed field free line by the apparatus may be an operation of obtaining identification information of the changed field free line. Herein, the identification information of the field free line may include information on the position of the field free line, a physical distance between the changed position and the initial position, a relation with the target, the strength of the magnetic fields, and a relation with the predetermined path.

More specifically, the apparatus may obtain a series of pieces of the identification information of the field free line that are generated while the field free line moves along the predetermined path. For example, the apparatus may obtain the pieces of the identification information of the field free line respectively corresponding to the initial position of the field free line, any position in the middle of the movement, and the final position after the movement ends.

In step S150, the apparatus generates image information on the target on the basis of the changed field free line.

The apparatus may obtain a detection signal on the basis of the field free line before and after change, and may process the detection signal to generate image information on the target. Herein, the image information may be referred to as MPI information, for example.

The operation of obtaining the detection signal on the basis of the field free line before and after change may include an operation of generating or extracting the detection signal by using the identification information corresponding to the field free line before and after change. Accordingly, the apparatus may generate image information on the basis of a detection signal obtained by measuring a field free line at a particular position.

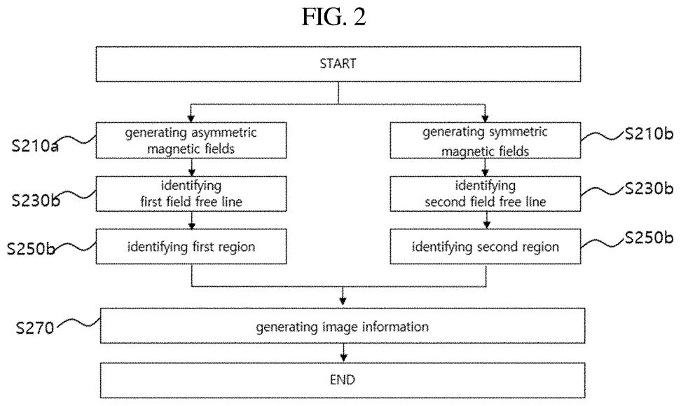

FIG. 2 is a flowchart illustrating another operation of an apparatus according to an embodiment of the present disclosure.

Referring to FIG. 2, an apparatus according to an embodiment of the present disclosure may include both an asymmetric magnetic field generating means and a symmetric magnetic field generating means. The asymmetric and/or symmetric magnetic field generating means may be composed of a plurality of independent magnetic pairs, and each of the magnetic parts may include a permanent magnet or an electromagnet. Hereinafter, the terms “magnetic part” and “magnetic member” may be interchangeably used.

With reference to FIG. 2, a case of asymmetric and symmetric magnetic field generating means each composed of two respective independent magnetic parts will be described as an example. The two magnetic parts constituting a magnetic field generating means may be referred to as a first magnetic part and a second magnetic part, respectively.

The asymmetric magnetic field generating means may generate magnetic force lines that form an asymmetric structure with respect to any reference line or reference plane. The magnetic force lines forming the asymmetric structure may be determined on the basis of differences in strength between the magnetic fields respectively generated from the first magnetic part and the second magnetic part. For example, when the magnetic force of the first magnetic part is greater than the magnetic force of the second magnetic part, the magnetic force lines formed by each of the magnetic parts may be biased toward the first magnetic part. That is, in this case, the magnetic field generating means may generate asymmetric magnetic fields.

In another example, when a magnetic field generating means includes three or more magnetic parts, asymmetric magnetic fields may be formed according to an arrangement structure of the first magnetic part, the second magnetic part, and the third magnetic part in addition to the strength of magnetic fields.

The symmetric magnetic field generating means may generate magnetic force lines that form a symmetric structure with respect to any reference line or reference plane. The magnetic force lines forming the symmetric structure may be formed when the magnetic fields respectively generated from the first magnetic part and the second magnetic part are the same in strength.

The apparatus may include both asymmetric and symmetric magnetic field generating means. In this case, the apparatus may obtain detection signals on a target simultaneously from the respective magnetic field generating means and may add up the detection signals to generate image information.

In another example, the apparatus may include one magnetic field generating means, and may change an arrangement structure or strength of magnetic fields of magnetic parts constituting the one magnetic field generating means, thereby realizing functions of both asymmetric and symmetric magnetic field generating means. In this case, the apparatus may provide a separate control command for changing the property of the magnetic field generating means. Herein, the control command for changing the property of the magnetic field generating means may be referred to as a second control command. In this case, a processor of the apparatus may generate the second control command and transmit the same to the magnetic field generating means, so that the magnetic fields generated by the magnetic field generating means are changed from symmetric magnetic fields to asymmetric magnetic fields or from asymmetric magnetic fields to symmetric magnetic fields.

According to an embodiment of the present disclosure, a process in which the apparatus generates image information on a target using each magnetic field generating means is as follows.

In step S210a, the apparatus generates asymmetric magnetic fields.

Specifically, the apparatus may activate the asymmetric magnetic field generating means to generate magnetic fields around the target. Herein, the generated magnetic fields may be formed asymmetrically with respect to a position of the target.

In step S230a, the apparatus identifies a first field free line.

The apparatus may identify a point or line at which the strength of the magnetic fields converges to 0 or is equal to or less than a predetermined reference value in the magnetic fields formed around the target. When two magnetic parts constituting a magnetic field generating means are provided symmetrically to each other and the magnetic parts generate symmetric magnetic fields, a field free line may be positioned at the center of the target. However, as in step S230a, when the magnetic parts forms asymmetric magnetic fields, the identified first field free line may be positioned at a point away from the center from the target. For example, the first field free line may be formed at a position away from the target to the left.

In step S250a, the apparatus identifies a first region.

The first region may be a virtual region formed by moving the first field free line along a predetermined path. More specifically, when the magnetic field generating means moves to the left or right according to a first control command received from the apparatus, the first field free line may also move in response to the movement of the magnetic field generating means. In this case, the first field free line may occupy the target or a predetermined region around the target, and the apparatus may identify the region as the first region.

The identifying of the first region by the apparatus may include an operation of obtaining a detection signal generated while the first field free line forms the first region.

In step S210b, the apparatus generates symmetric magnetic fields.

Specifically, the apparatus activates the symmetric magnetic field generating means to generate magnetic fields around the target. Herein, the generated magnetic fields may be formed symmetrically with respect to a position of the target.

In step S230b, the apparatus identifies a second field free line.

The apparatus may identify a point or line at which the strength of the magnetic fields converges to 0 or is equal to or less than a predetermined reference value in the magnetic fields formed around the target. When two magnetic parts constituting a magnetic field generating means are provided symmetrically to each other and the magnetic parts generate symmetric magnetic fields, a field free line may be positioned at the center of the target.

In step S250b, the apparatus identifies a second region.

The second region may be a virtual region formed by moving the second field free line along a predetermined path. More specifically, when the magnetic field generating means moves to the left or right according to a first control command received from the apparatus, the second field free line may also move in response to the movement of the magnetic field generating means. In this case, the second field free line may occupy the target or a predetermined region around the target, and the apparatus may identify the region as the second region.

The identifying of the second region by the apparatus may include an operation of obtaining a detection signal generated while the second field free line forms the second region.

In step S270, the apparatus generates image information.

The apparatus may generate the image information on the basis of the first region and the second region. More specifically, one piece of image information on the target may be generated by analyzing a detection signal related to the target from each region excluding the overlapping region between the first region and the second region.

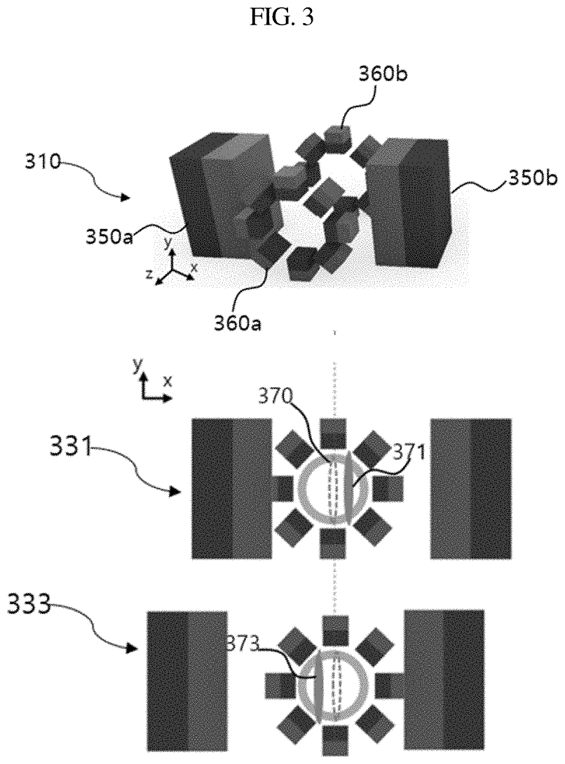

FIG. 3 is a configuration diagram illustrating a magnetic field generating means included in an apparatus according to an embodiment of the present disclosure.

Referring to FIG. 3, the apparatus according to an embodiment of the present disclosure includes a symmetric magnetic field generating means. More specifically, a magnetic field generating means 310 includes a first magnetic part 350a and a second magnetic part 350b that are provided symmetrically with respect to the z axis, and includes a third magnetic part 360a and a fourth magnetic part 360b that are provided symmetrically with respect to the x axis.

A first plane view 331 and a second plane view 333 showing the magnetic field generating means 310 on the xy plane illustrate an operation in which a field free line moves in response to obtaining a first control command of the magnetic field generating means.

In the first plane view 331, the magnetic field generating means may move to the right in response to the first control command. In this case, a 1-1 field free line 370 formed at the middle of the magnetic field generating means may move to the right by a predetermined distance. The apparatus may identify a 1-2 field free line 371, which is the field free line resulting from change according to the movement of the magnetic field generating means after a 1-1 control command is provided.

The region from the position of the 1-1 field free line 370 to the position of the 1-2 field free line resulting from change according to the first control command may be defined as a 1-1 region.

In the second plane view 333, the magnetic field generating means may move to the left in response to a 1-2 control command. In this case, the 1-1 field free line 370 formed at the middle of the magnetic field generating means may move to the left by a predetermined distance. The apparatus may identify a 1-3 field free line 373, which is the field free line resulting from change according to the movement of the magnetic field generating means after the 1-2 control command is provided.

The region from the position of the 1-1 field free line 370 to the position of the 1-3 field free line resulting from change according to the 1-2 control command may be defined as a 1-2 region.

The apparatus may obtain region information corresponding to the 1-1 region and the 1-2 region (hereinafter, referred to as a “first region”), and may generate image information on the basis of the obtained region information. However, as shown in FIG. 3, when the magnetic field generating means is symmetric, a field free line may not be measured in a region beyond a threshold distance from the 1-1 field free line. Therefore, the apparatus needs to generate image information on a target, depending on the region information corresponding to the first region.

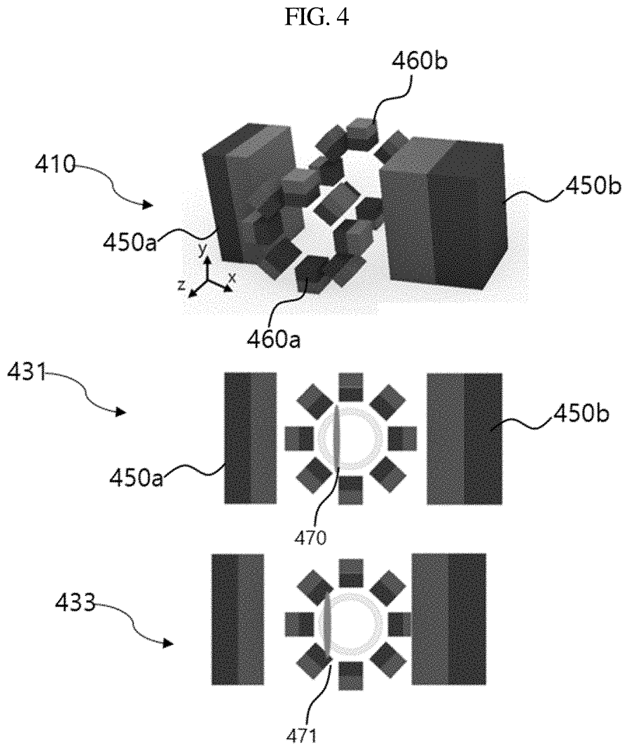

FIG. 4 is a configuration diagram illustrating a magnetic field generating means included in an apparatus according to an embodiment of the present disclosure.

Referring to FIG. 4, the apparatus according to an embodiment of the present disclosure may include an asymmetric magnetic field generating means. More specifically, a magnetic field generating means 410 includes a fifth magnetic part 450a and a sixth magnetic part 450b that are provided symmetrically with respect to the z axis, and includes a seventh magnetic part 460a and an eighth magnetic part 460b that are provided symmetrically with respect to the x axis.

A first plane view 431 and a second plane view 433 showing the magnetic field generating means 410 on the xy plane illustrate an operation in which a field free line moves in response to obtaining a first control command of the magnetic field generating means.

The first plane view 431 shows a position of a 2-1 field free line 470 before the magnetic field generating means obtains a first control command. The 2-1 field free line 470 may be positioned close to the left by a predetermined distance from the middle of the magnetic field generating means. Preferably, the position of the 2-1 field free line 470 close to the left may be the same as the left than the position of the 1-3 field free line 373 at the maximum distance by which the symmetric magnetic field generating means shown in FIG. 3 moves to the left. Alternatively, the position of the 2-1 field free line 470 may be closer to the left than the position of the 1-3 field free line 373 is.

In the second plane view 433, the magnetic field generating means may move to the left in response to the first control command. In this case, the 2-1 field free line 470 may move to the left by a predetermined distance. The apparatus may identify a 2-2 field free line 471, which is the field free line resulting from change according to the movement of the magnetic field generating means after the first control command is provided.

The region from the position of the 2-1 field free line 470 to the position of the 2-2 field free line resulting from change according to the first control command may be defined as a second region.

The apparatus may obtain region information corresponding to the second region, and may generate image information on the basis of the obtained region information. The second region may include a new detection signal related to the target within a range that does not overlap with the first region identified in FIG. 3. Accordingly, the apparatus may obtain more extended image information on a target by using both symmetric and asymmetric magnetic field generating means.

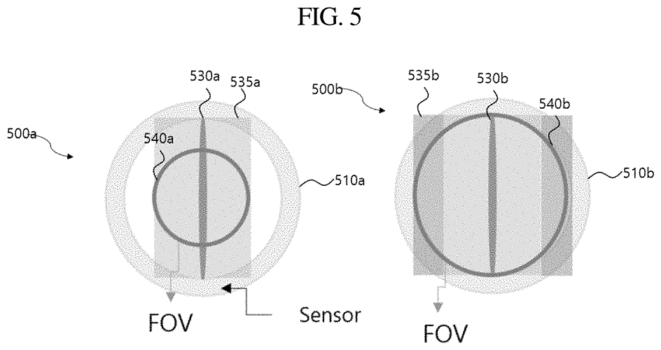

FIG. 5 is a configuration diagram illustrating an operation of generating image information with an extended FoV by an apparatus according to an embodiment of the present disclosure.

Referring to FIG. 5, an internal configuration diagram of magnetic field generators shows a magnetic field generator 500a for generating symmetric magnetic fields and a magnetic field generator 500b for generating asymmetric magnetic fields in the apparatus.

In an environment in which the apparatus generates symmetric magnetic fields, when a magnetic field generating means moves according to a first control command, a field free line 530a also moves in response to the movement of the magnetic field generating means and may thus occupy a predetermined region 535a. Herein, the region that the field free line 530a occupies may be referred to as a first region. The apparatus may generate image information on the basis of region information on the first region. The FoV of the generated image information may be related to the maximum size of a circle 540a that may be included in the first region.

In an environment in which the apparatus generates asymmetric magnetic fields, when a magnetic field generating means moves according to a first control command, a field free line 530b also moves in response to the movement of the magnetic field generating means and may thus occupy a predetermined region 535b. Herein, the region that the field free line 530b occupies may be referred to as a second region.

The apparatus may generate image information on the basis of region information on a region that is the sum of the first region 535a and the second region 535b. That is, the FoV of the generated image information may be related to the maximum size of a circle 540b that may be included in the first region and the second region.

Accordingly, the apparatus is capable of extending the FoV of image information to a region that a symmetric magnetic field generator is unable to identify, by using a field free line identified by an asymmetric magnetic field generating means.

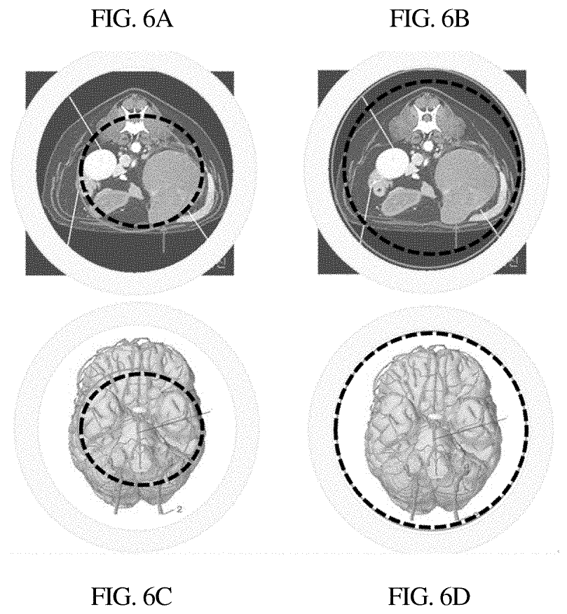

FIGS. 6A to 6D are diagrams illustrating image information generated by an apparatus according to an embodiment of the present disclosure.

The apparatus according to an embodiment of the present disclosure uses an asymmetric magnetic field generating means to obtain information on a field free line that a symmetric magnetic field generating means is unable to identify, thereby extending the FoV of image information on a target.

Specifically, in MPI of the human abdomen or brain, if an MPI device with a symmetric magnetic field generating means is used, the outer region of the abdomen or the outer blood vessel portion of the brain may be missed.

In this case, using an asymmetric magnetic field generating means together extends a region across which a field free line passes, extending the FoV of a generated MPI image.

It will be understood by those skilled in the art that modifications to the embodiments may be made without departing from the essential features of the disclosure. Thus, the described embodiments should be considered in a descriptive sense only and not for purposes of limitation. Therefore, the scope of the disclosure is defined not by the detailed description of the disclosure but by the following claims, and all variations within the scope of the claims and their equivalents are to be construed as being included in the embodiments.

What is claimed is:

1. An apparatus for obtaining image information on a target using magnetic particle imaging (MPI), the apparatus comprising
a magnetic field generating means comprising a first magnetic member and a second magnetic member; and
at least one processor operably connected to the magnetic field generating means,
wherein the at least one processor is configured to
cause the magnetic field generating means to form magnetic fields in an ambient space of the target according to a predetermined rule,
determine, as a field free line (FFL) a position corresponding to a point, a line, or a plane at which strength of the magnetic fields in the ambient space is less than a threshold value,
provide a first control command for the magnetic field generating means such that the field free line moves along a predetermined path,
identify the field free line changed in response to movement of the magnetic field generating means according to the first control command, and
generate the image information on the target on the basis of the field free line changed, and
the magnetic fields generated from the first magnetic member and the second magnetic member are asymmetric with respect to the target.

2. The apparatus of claim 1, wherein the predetermined path is a first path formed asymmetrically with respect to the target.

3. The apparatus of claim 1, wherein the at least one processor is further configured to
provide a second control command for control such that positions of the first magnetic member and the second magnetic member are exchanged or strengths of the magnetic fields generated by the first magnetic member and the second magnetic member are exchanged, and
the field free line changed is identified in response to movement of the magnetic field generating means according to the second control command.

4. The apparatus of claim 1, wherein the magnetic fields generated from the first magnetic member and the second magnetic member are generated symmetrically with respect to the target, and
the predetermined path is a second path formed symmetrically with respect to the target.

5. The apparatus of claim 2, wherein the at least one processor is configured to
identify a final field free line on the basis of the field free line moving along the first path or the field free line moving along the second path, and
generate the image information on the target on the basis of the final field free line.

6. An operation method of an apparatus for obtaining image information on a target using magnetic particle imaging (MPI), the operation method comprising:
causing a magnetic field generating means to form magnetic fields in an ambient space of the target according to a predetermined rule;
determining, as a field free line (FFL), a position corresponding to a point, a line, or a plane at which strength of the magnetic fields in the ambient space is less than a threshold value;
providing a first control command for the magnetic field generating means such that the field free line moves along a predetermined path;
identifying the field free line changed in response to movement of the magnetic field generating means according to the first control command; and
generating the image information on the target on the basis of the field free line changed,
wherein the magnetic fields respectively generated from a first magnetic member and a second magnetic member that the magnetic field generating means comprises are asymmetric with respect to the target.

7. The operation method of claim 6, wherein the predetermined path is a first path formed asymmetrically with respect to the target.

8. The operation method of claim 6, further comprising
providing a second control command for control such that positions of the first magnetic member and the second magnetic member are exchanged or strengths of the magnetic fields generated by the first magnetic member and the second magnetic member are exchanged,
wherein the field free line changed is identified in response to movement of the magnetic field generating means according to the second control command.

9. The operation method of claim 6, wherein the magnetic fields generated from the first magnetic member and the second magnetic member are generated symmetrically with respect to the target, and
the predetermined path is a second path formed symmetrically with respect to the target.

10. The operation method of claim 7, further comprising
identifying a final field free line on the basis of the field free line moving along the first path or the field free line moving along the second path; and
generating the image information on the target on the basis of the final field free line.

11. An apparatus for obtaining image information on a target using magnetic particle imaging (MPI), the apparatus comprising
a first magnetic field generating means provided symmetrically with respect to the target;
a second magnetic field generating means provided asymmetrically with respect to the target; and
at least one processor operably connected to the first magnetic field generating means and the second magnetic field generating means,
wherein the at least one processor is configured to
cause the first magnetic field generating means and the second magnetic field generating means to form magnetic fields in an ambient space of the target according to a predetermined rule,
determine, as field free lines (FFLs), positions corresponding to points, lines, or planes at which strengths of the magnetic fields in the ambient space are less than a threshold value, the field free lines including a first field free line and a second field free line respectively corresponding to the first magnetic field generating means and the second magnetic field generating means,
identify, as a first region and a second region, respective regions that the first field free line and the second field free line occupy as moving along respective predetermined paths, and
generate the image information on the target on the basis of the identified first region and the identified second region.

12. The apparatus of claim 11, wherein the predetermined paths comprise:
a first path formed symmetrically with respect to the target; and
a second path formed asymmetrically with respect to the target,
wherein the first path corresponds to the first region, and the second path corresponds to the second region.

13. The apparatus of claim 11, wherein the at least one processor is configured to
identify an overlapping region between the first region and the second region, and
exclude the overlapping region in order to generate the image information on the target on the basis of the identified first region and the identified second region.

14. The apparatus of claim 4, wherein the at least one processor is configured to
identify a final field free line on the basis of the field free line moving along the first path or the field free line moving along the second path, and
generate the image information on the target on the basis of the final field free line.

15. The operation method of claim 9, further comprising:
identifying a final field free line on the basis of the field free line moving along the first path or the field free line moving along the second path; and
generating the image information on the target on the basis of the final field free line.
